(19) United States
(12) Patent Application Publication
(10) Pub. No.: US 2024/0165324 A1
(43) Pub. Date: May 23, 2024

(54) INFUSION MANAGEMENT SYSTEM

(71) Applicant: CareFusion 303, Inc., San Diego, CA (US)

(72) Inventors: Daniel M. Abal, San Diego, CA (US); John Denuzzio, La Jolla, CA (US); Brendan Burgess, Poway, CA (US)

(21) Appl. No.: 18/549,831

(22) PCT Filed: Mar. 10, 2022
(86) PCT No.: PCT/US2022/019803

§ 371 Date: Sep. 8, 2023

Related U.S. Application Data
(60) Provisional application No. 63/159,929, filed on Mar. 11, 2021.

(51) Int. Cl. A61M 5/142

(52) U.S. Cl. CPC A61M 5/14244; A61M 2005/14268; A61M 2205/18; A61M 2205/3584; A61M 2205/502

(57) ABSTRACT

An infusion pump system may include a portable infusion pump configured to be worn by a user and a separable display. The portable infusion pump may include a fluid container configured to store a fluid to be delivered to the user, a pumping mechanism configured to drive the delivery of the fluid from the fluid container to the user via a fluid delivery tube, and communications circuitry. The separable display may transmit, to the communications circuitry of the portable infusion pump when the separable display is coupled to the portable infusion pump, a fluid delivery protocol. The separable display may be separated from the portable infusion pump after transmission of the fluid delivery protocol to the portable infusion pump.

### CROSS-REFERENCE TO RELATED APPLICATIONS

The present application claims priority to U.S. Provisional Application No. 63/159,929, filed Mar. 11, 2021, and entitled, “Infusion Management System,” the entirety of which is incorporated by reference herein.

### TECHNICAL FIELD

The subject matter described herein relates generally to the dispensation of medications and more specifically to an infusion management system for the delivery of medication to a patient.

### BACKGROUND

Fluid pumps, such as infusion pumps, administer therapy to patients by delivering a medication or other fluid to the patient. In some instances, a particular type of therapy, such as chemotherapy, takes a prolonged amount of time to deliver the medication to the patient. The fluid pumps may be large, making it difficult to store and/or use the pumps, as the available space in a medical facility or patient care room may be limited, and in some instances may require the caregiver to control the pump for the duration of the delivery of therapy. Additionally and/or alternatively, certain types of therapy, such as the delivery of insulin or chemotherapy, may be delivered to the patient at a location other than the medical facility, such as at the patient's home, or while the patient is traveling. Also, certain clinical treatments may require patients to go to a medical facility for administration of the therapy multiple days over an extended period of time. However, administering therapy to patients at locations other than at the medical facility may be difficult due to the poor portability of the pumps and the inability for the pumps to be remotely controlled and/or programmed. Providing relevant information to the patient or caregiver about the pump or the administered therapy may also be difficult.

### SUMMARY

Systems, methods, and articles of manufacture, including computer program products, are provided for an infusion management system for dispensing a medication to a patient. For example, the system may provide an infusion pump system including a portable infusion pump that may be easily carried and/or worn by the user. The infusion pump system may include the portable infusion pump and a display that is separable from the portable infusion pump. The separable display may configure the portable infusion pump with a fluid delivery protocol. The portable infusion pump may additionally and/or alternatively be disposable.

According to some aspects, an infusion pump system includes a portable infusion pump and/or a separable display. The portable infusion pump may be worn by a user. The portable infusion pump may include a fluid container configured to store a fluid to be delivered to the user, a pumping mechanism configured to drive the delivery of the fluid from the fluid container to the user via a fluid delivery tube, and communications circuitry that includes an antenna. The separable display may transmit, to the communications circuitry of the portable infusion pump when the separable display is coupled to the portable infusion pump, a fluid delivery protocol. The separable display may be separated from the portable infusion pump after transmission of the fluid delivery protocol to the portable infusion pump. The fluid delivery protocol may define one or more parameters for delivering the fluid to the user.

In some aspects, the portable infusion pump is disposable.

In some aspects, the communications circuitry includes at least one of a low energy communications circuitry, a near field communications circuitry, and a Bluetooth low energy communications circuitry.

In some aspects, the separable display includes an attachment sensor. The separable display may transmit the fluid delivery protocol when a signal from the attachment sensor indicates that the separable display is physically coupled to the portable infusion pump.

In some aspects, the physical coupling includes a magnetic coupling.

In some aspects, the separable display includes a proximity sensor. The separable display may transmit the fluid delivery protocol when a signal from the proximity sensor indicates that the separable display is positioned within a predetermined distance from the portable infusion pump.

In some aspects, the infusion pump system includes the fluid delivery tube.

In some aspects, the portable infusion pump includes a waterproof housing. At least the fluid container, the pumping mechanism, and the communication circuitry may be disposed within the waterproof housing.

In some aspects, the portable infusion pump includes an alert system for generating an alert upon detection of an error during the delivery of the fluid to the user. The alert may include one or more of an audio, visual, and tactile alert.

In some aspects, the fluid container is configured to hold less than or equal to 100 milliliters of the fluid.

According to some aspects, a method includes receiving, by a portable infusion pump from a display, at least one fluid delivery protocol. The display may be temporarily coupled to the portable infusion pump. The method may also include delivering, by the portable infusion pump and based on the at least one fluid delivery protocol, a fluid to a user. The method may also include measuring, by the portable infusion pump, one or more fluid delivery parameters associated with the delivery of the fluid. The method may also include establishing, by the portable infusion pump, a wireless connection with the display. The method may also include transmitting, by the portable infusion pump to the display in wireless communication with the portable infusion pump, the one or more fluid delivery parameters.

In some aspects, the method also includes detecting an error in the delivery of the fluid to the user and stopping the delivery of the fluid to the user.

In some aspects, the method also includes transmitting the error to the display when the display is in wireless communication with the portable infusion pump, and displaying information associated with the error via the display.

In some aspects, the portable infusion pump includes a fluid container configured to store the fluid to be delivered to the user, a pumping mechanism configured to drive the delivery of the fluid from the fluid container to the user via a fluid delivery tube, and communications circuitry including an antenna.

In some aspects, the method also includes establishing, by the portable infusion pump, a wireless connection with the display when an attachment sensor of the display indicates that portable infusion pump is physically coupled to the display. The method may also include disconnecting, by the portable infusion pump, the wireless connection with the display when the portable infusion pump is not physically coupled to the display.

In some aspects, the portable infusion pump is disposable.

In some aspects, the communications circuitry includes at least one of a low energy communications circuitry, a near field communications circuitry, and a Bluetooth low energy communications circuitry.

In some aspects, the portable infusion pump further includes a fill port, through which the fluid container is configured to be filed.

In some aspects, the fluid container is configured to hold a plurality of fluid bags.

In some aspects, the portable infusion pump includes a waterproof housing, and at least the fluid container, the pumping mechanism, and the communication circuitry are disposed within the waterproof housing.

In some aspects, the method also includes the portable infusion pump further includes an alert system for generating an alert upon detection of an error during the delivery of the fluid to the user. The alert may include one or more of an audio, visual, and tactile alert.

In some aspects, the fluid container is configured to hold less than or equal to 100 milliliters of the fluid.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein.

Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to an infusion management system, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an infusion management system, in accordance with some example embodiments;

FIG. 2 schematically depicts an infusion pump system, in accordance with some example embodiments;

FIG. 3A depicts an example infusion pump system including a portable infusion pump and a display, in accordance with some example embodiments;

FIG. 3B depicts an example infusion pump system including a portable infusion pump and a display, in accordance with some example embodiments;

FIG. 3C depicts a user device and an example infusion pump system including a portable infusion pump and a display, in accordance with some example embodiments;

FIG. 4A depicts an example portable infusion pump, in accordance with some example embodiments;

FIG. 4B depicts an example infusion pump system including a portable infusion pump and a display, in accordance with some example embodiments;

FIG. 4C depicts an example infusion pump system including a portable infusion pump and a user device, in accordance with some example embodiments;

FIG. 4D schematically depicts an example portable infusion pump, in accordance with some example embodiments;

FIG. 5 depicts a flowchart illustrating a process for configuring a portable infusion pump, in accordance with some example embodiments;

FIG. 6 depicts a block diagram illustrating a computing system, in accordance with some example embodiments;

FIG. 7A depicts a front view of a patient care system, in accordance with some example embodiments;

FIG. 7B depicts an enlarged view of a portion of a patient care system, in accordance with some example embodiments; and

FIG. 7C depicts a perspective view of a pump, in accordance with some example embodiments.

When practical, similar reference numbers denote similar structures, features, or elements.

### DETAILED DESCRIPTION

Fluid pumps, such as infusion pumps, administer therapy to patients by delivering a medication or other fluid to the patient. The delivery of certain therapies, such as chemotherapy, may take a prolonged amount of time, requiring the patient to go to a medical facility every day and/or requiring the caregiver to stay with the patient throughout the duration of the delivery of therapy to the patient. The fluid pumps may be very large, occupy a large amount of space within a hospital and/or patient care room, and may be very cumbersome to use. For example, during cancer treatments, the administration of chemotherapy may be combined with one or more other treatments, such as hydration treatments. The infusion management system described herein includes one or more portable infusion pumps, which may be smaller than the pumps (e.g., multi-channel pumps) generally used for delivering certain therapies, such as chemotherapy or other therapies during which multiple types of fluid therapies may be combined. For example, the infusion pump system described herein may include a modular system including a separable portable infusion pump and a display. In some implementations, the portable infusion pump may be disposable. By reducing the size of the portable infusion pumps, a greater number of pumps may be stored within a medical facility and/or patient care room, making it easier to deliver therapies that include the delivery of multiple types of fluid, and improving the flexibility in these types of treatments.

Reducing the size of the pumps, such as by providing the portable infusion pump described herein, may allow the pumps to be located adjacent to and/or close to the infusion site on the patient's body. This reduces the risk of incorrect and/or displaced connection in the fluid delivery tubes connecting the portable infusion pump to the patient's body, as the pump is located close to the infusion site. This may also reduce influence of the fluid delivery line on the administration of the fluid therapy to the patient. As a result, the portable infusion pumps described herein may improve the accuracy of measurements made based on the delivered therapy.

Additionally and/or alternatively, certain types of therapy, such as the delivery of insulin or chemotherapy, may be delivered to the patient at a location other than at the medical facility, such as at the patient's home, or while the patient is traveling or over a prolonged period of time at a medical facility, such as with caregiver assistance. As noted above, fluid pumps may generally be very large, making it difficult to store and/or use the pumps. Thus, administering therapy to patients at locations other than at the medical facility may be difficult due to the poor portability of the pumps and the inability for the pumps to be remotely controlled and/or programmed. Providing relevant information to the patient about the pump or the administered therapy may be difficult. The infusion management system including the portable infusion pumps described herein may allow patients to receive fluid therapy in their homes, rather than at a medical facility, leading to a more comfortable life, and improved experience receiving the fluid therapy. The portable infusion pumps described herein may also be worn on a patient's body and/or easily carried by the patient, allowing the patient to receive the administered therapy at various locations. The portable infusion pumps may also allow patients to engage in normal activities, such as traveling, sleeping, and/or showering, without interrupting the delivery of therapy. As such, the portable infusion pumps described herein may provide the same functionality as the pumps provided at the medical facility, yet allow the patients to lead their normal lifestyles with no or minimal encumbrance or the portable infusion pumps being readily noticeable.

In some implementations, the portable infusion pumps described herein may also be remotely controlled and/or programmed by an application on a mobile device or standalone display and/or transmit useful data about the administered therapy to the display to be displayed to the patient and/or be transmitted to the medical facility. Such configurations may make it easier for the patients (or caregivers thereof) to use the portable infusion pumps to administer the fluid therapy, may help to reduce the size of the portable infusion pumps by eliminating the need for a display on the pumps carried by the patients, and may help to improve the portability of the portable infusion pumps. The portable infusion pumps described herein may also allow caregivers to monitor the delivery of the therapy to the patient. For example, if the portable infusion pump detects an issue that impacts the delivery of the therapy, such as an IV line occlusion, the portable infusion pump may send an alert to the caregiver and/or the patient to correct the issue. This configuration may be helpful in circumstances, such as when access to caregivers is restricted.

Consistent with implementations of the current subject matter, the portable infusion pumps may additionally and/or alternatively have a reduced cost, as the pumps may include a reduced set of components (e.g., a separable display and/or a disposable pump). Such configurations may provide a consolidated interface that is easier to use for the patient. The consolidated interface (e.g., via the separate display or user device) may be easier to use and/or read by the patient. Rules may be implemented using the consolidated interface to prevent the administration of incompatible fluids to the patient. Additionally and/or alternatively, the portable infusion pump may allow for the patient to program or otherwise set up the portable infusion pump without or with minimal caregiver supervision.

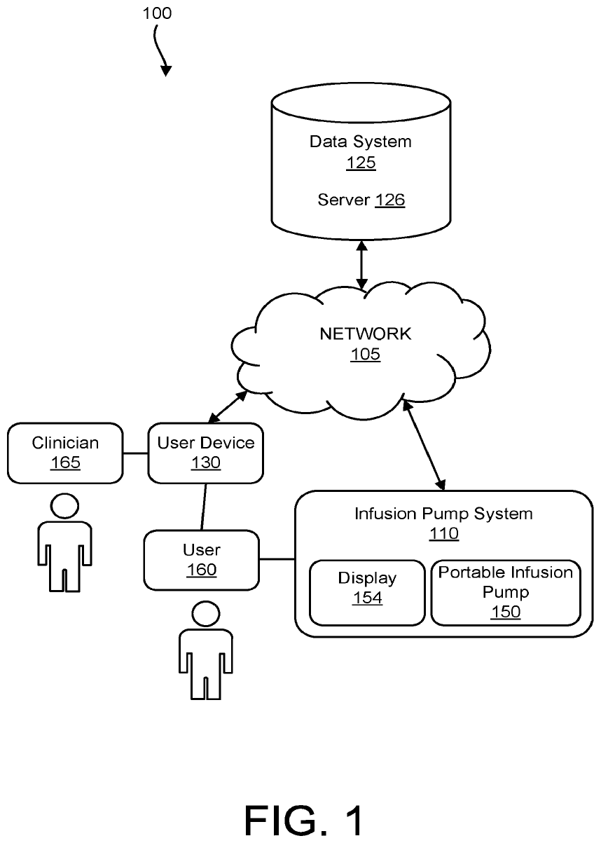

FIG. 1 depicts a system diagram illustrating an infusion management system 100, in accordance with some example implementations. Referring to FIG. 1, the infusion management system 100 may include an infusion pump system 110 including a portable infusion pump 150 (also referred to herein as an “infusion device”) and a display 154, a user device 130, and a data system 125.

The user device 130 may be a mobile device such as, for example, a smartphone, a tablet computer, a wearable apparatus, and/or the like. However, it should be appreciated that the user device 130 may be any processor-based device including, for example, a desktop computer, a laptop computer, a workstation, a network-connected television, and/or the like. For example, via the user device 130, the user 160 may be able to view certain parameters of the pump 150, such as information relating to the user, the fluid therapy being delivered, and/or an alert generated based on the pump 150. The information relating to the fluid therapy being delivered may include a time or time interval of the fluid therapy delivery, an amount of fluid being delivered, a type of fluid being delivered, and/or the like. The user device 130 may additionally or alternatively be used by a clinician 165 (also referred to herein as a “caregiver”) and/or the user 160 to configure certain parameters of the pump 150, such as a time or time interval of the fluid therapy delivery, an amount of fluid being delivered, a type of fluid being delivered, and/or the like. Additionally, in some examples, via the user device 130, the clinician 165 may configure various fluid delivery protocols with default settings and safety parameters (e.g., setting a limit to a dose of a delivered fluid).

In some implementations, the user device 130 may directly program the pump 150 with the fluid delivery protocol, transmit data to the pump 150, and/or receive data from the pump 150. For example, the pump 150 may read from and/or write to a data tag of the display 154 and/or the user device 130 when the pump 150 is positioned away from the data tag within a predetermined distance and/or for a predetermined amount of time. The predetermined distance may be approximately 3 cm. In some embodiments, the predetermined distance is approximately 1 to 2 cm, 2 to 3 cm, 3 to 4 cm, and/or other ranges therebetween. In other embodiments, the pump 150 may be held in contact with the data tag, the display 154, and/or the user device 130. The predetermined amount of time may be 0.5 seconds to 1 second. In some embodiments, the predetermined amount of time is approximately 0.5 to 1.5 seconds, 1.0 to 2.0 seconds, 2.0 seconds to 3.0 seconds, 3.0 seconds to 4.0 seconds, and/or the like. Limiting reading from and/or writing to the data tag of the display 154 and/or the user device 130 to when the pump 150 is positioned away from the data tag within the predetermined distance and/or for the predetermined amount of time helps to improve security by reducing or eliminating the likelihood of a security breach.

The data system 125 may include one or more databases, providing physical data storage within a dedicated facility and/or being locally stored on the pump 150 and/or the display 154. The data system 125 may include an inventory system, a patient care system, an administrative system, an electronic medical record system, and/or the like, which store a plurality of electronic medical records, each of which include the patient's medical history, one or more fluid delivery protocols, and/or the like. Additionally and/or alternatively, the data system 125 may include cloud-based systems providing remote storage of data in, for example, a multi-tenant computing environment and/or the like. The data system 125 may also include non-transitory computer readable media.

In some implementations, the data system 125 may include and/or be coupled to a server 126, which may be a server coupled to a network, a cloud server, and/or the like. The user device 130 may wirelessly communicate with the server 126. The server 126 may communicate directly with the pump 150, or through the user device 130 and/or the display 154. The server 126, which may include a cloud-based server, may provide data and/or instructions from the data system 125 to the pump 150 and/or the user device 130 or display 154 to be transmitted to the pump 150, to implement one or more features of the fluid therapy protocols consistent with implementations of the current subject matter. Additionally and/or alternatively, the server 126 may receive data (e.g., one or more parameters of the fluid therapy) from the user device 130 and/or the display 154 that has been transmitted to the user device 130 and/or the display 154 from the pump 150.

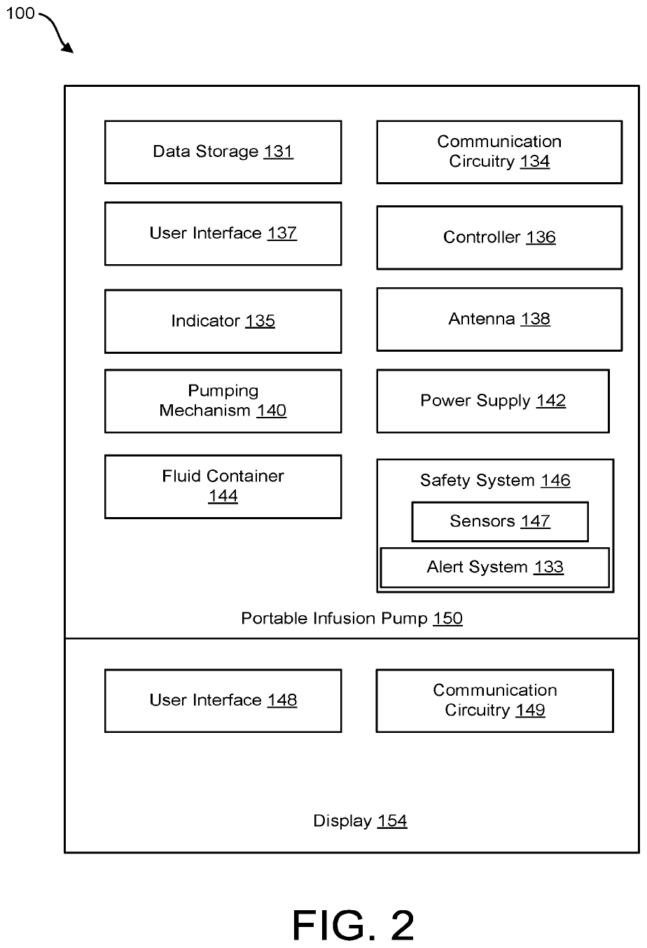

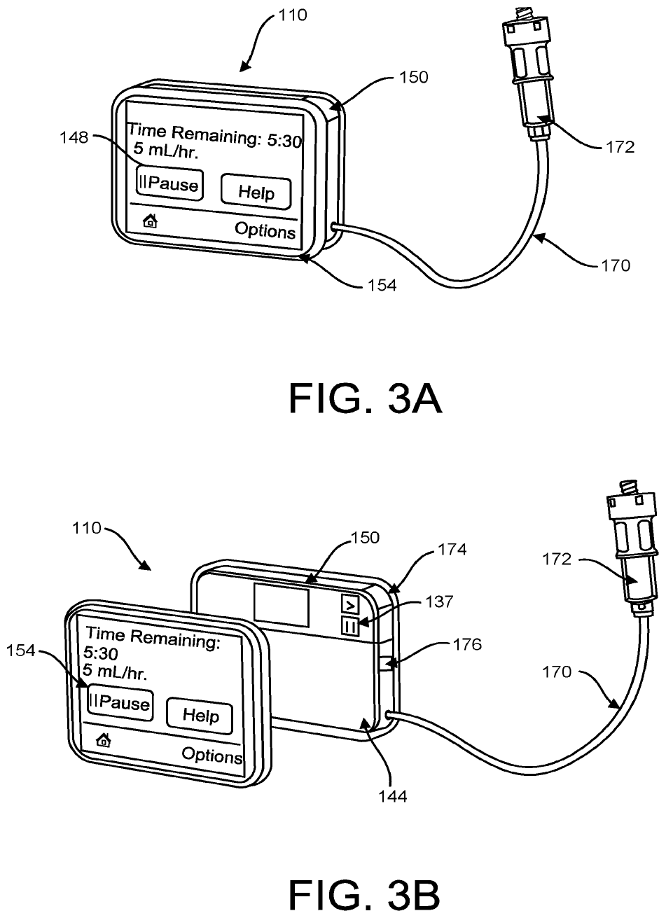

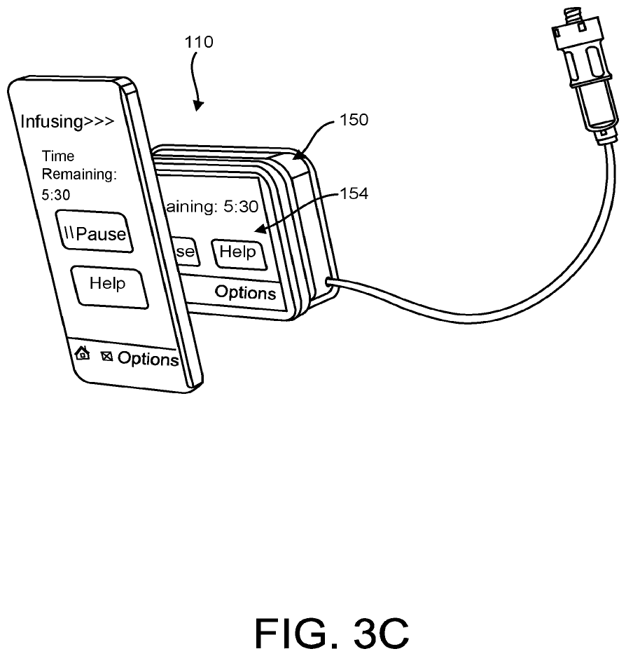

An application software (“app”) running on at least one of the user device 130 and/or the display 154 may be configured to control operational aspects of the pump 150, and receive information relating to operation of the pump 150. For example, the app may provide the user 160 with a limited set of functionalities, such as viewing a status of the pump 150, one or more parameters of the fluid therapy being delivered by the pump 150, an error in the administration of the fluid therapy, and/or the like. The app may display, via a user interface 148 (see FIGS. 2 and 3A), one or more parameters of the fluid therapy, such as a fluid delivery rate, a Volume to Be Infused (VTBI), and/or the like, a battery status showing the status of the power supply 142 of the pump 150, an amount of fluid remaining in the fluid container 144 of the pump 150, and/or the like. In some implementations, the app may additionally and/or alternatively provide the clinician 165 with capabilities to, via the app, retrieve one or more fluid delivery protocols from the server 126, establish a secure connection between the app, the display 154, and/or the pump 150, and transmit the fluid delivery protocols to the pump 150. The app may also provide the clinician 165 with functionality to select, such as via the user interface 148, one or more sets of properties or parameters of the fluid delivery protocols transmitted to the pump 150. In other words, the user interface 148 may detect a user interaction including a selection of the one or more sets of properties or parameters of the fluid delivery protocols transmitted to the pump 150. In some implementations, as described in more detail below, the app may log various parameters of the fluid therapy delivered by the pump 150, and transmit the logged parameters to the server 126.

Additionally and/or alternatively, the app of the user device 130 and/or the display 154 may be operated by the clinician 165 to retrieve the one or more fluid delivery protocols from the server 126. For example, the app and/or the display 154 may detect a user interaction, such as a request to retrieve the one or more fluid delivery protocols from the server 126 and/or a selection of one or more fluid delivery protocols. In some implementations, the app may include one or more authentication features to allow the clinician 165 access to the server 126, and to prevent the user 160 from accessing certain features (e.g., the retrieval and/or altering of a fluid delivery protocol) of the app. For example, the app may prompt, via the user interface of the user device 130 and/or the user interface 148 of the display 154, the clinician 165 to enter a user name and password, scan a badge, perform a fingerprint scan or a retina scan, and/or use facial recognition to identify the clinician 165, to provide access, via the app, to retrieve and/or alter one or more aspects of the fluid delivery protocols.

The infusion pump system 110 including the display 154 and/or the pump 150, the user device 130, and/or the data system 125 may be communicatively coupled to one another via a network 105. The network 105 may be a wired and/or wireless network including, for example, a public land mobile network (PLMN), a local area network (LAN), a virtual local area network (VLAN), a wide area network (WAN), the Internet, a short range radio connection, for example Bluetooth, a peer-to-peer mesh network, and/or the like.

FIG. 2 schematically depicts the infusion pump system 110, consistent with embodiments of the current subject matter. The infusion pump system 110 may include the portable infusion pump 150 and the display 154. The display 154 may be separable from the pump 150. For example, the display 154 may communicatively and/or physically couple with the pump 150 to program and/or transmit data to the pump 150 and/or to receive data from the pump 150. In some implementations, the display 154 physically couples to the pump 150 via a magnetic connection, snap-fit arrangement, friction fit arrangement, mechanical fastener, and/or the like. The display 154 and the pump 150 may have a same or similar size and/or shape. For example, the display 154 may have an outer perimeter and/or a shape that is the same as or similar to an outer perimeter and/or a shape of the pump 150. This allows for the pump 150 and display 154 to be easily held and/or coupled to one another during programming of the pump 150 and/or transmission of the data to and/or from the pump 150.

In some implementations, as described herein, the display 154 may program the pump 150 when the display 154 is communicatively and/or physically coupled to the pump 150. For example, the display may transmit information, such as a fluid delivery protocol, to the pump 150 when the display is communicatively and/or physically coupled to the pump 150. The display 154 may be separated and/or otherwise removed from the pump 150, such as after the transmission of data to and/or from the pump 150. This reduces the weight of the pump system 110 and allows the pump 150 to be easily carried and/or worn by the patient, such as during the delivery of the therapy to the patient.

In some implementations, the display 154 includes a sensor, such as an attachment sensor, a proximity sensor, and/or the like. The sensor may detect when the display 154 is within the predetermined distance from the pump 150. For example, in some implementations, the display 154 may transmit the data to and/or from the pump 150 when a signal from the attachment sensor indicates that the display 154 is coupled (e.g., physically coupled) to the pump 150. Additionally and/or alternatively, the display 154 may transmit the data to and/or from the pump 150 when a signal from the proximity sensor indicates that the display 154 is coupled (e.g., communicatively coupled) with the pump 150 and/or is positioned within a predetermined distance from the pump 150.

The pump 150 may be a TCI pump, a syringe pump, an anesthesia delivery pump, a patient-controlled analgesic (PCA) pump, a large volume pump (LVP), a small volume pump (SVP), and/or the like, configured to deliver a medication to a patient. However, it should be appreciated that the pump 150 may be any infusion device configured to deliver a substance (e.g., fluid, nutrients, medication, and/or the like) to a patient's circulatory system or epidural space via, for example, intravenous infusion, subcutaneous infusion, arterial infusion, epidural infusion, and/or the like. Alternatively, the pump 150 may be an infusion device configured to deliver a substance (e.g., fluid, nutrients, medication, and/or the like) to a patient's digestive system via a nasogastric tube (NG), a percutaneous endoscopic gastrostomy tube (PEG), nasojejunal tube (NJ), and/or the like. Moreover, the pump 150 may be part of a patient care system that includes one or more additional pumps.

The pump 150 may have limited functionality and/or features usable by the user 160. As noted above, the display 154 is separable from the pump 150 to allow the pump to be carried and/or worn by the patient in use. The pump 150 may include a user interface 137, a data storage 131, a pumping mechanism 140, a fluid container 144, communication circuitry 134, an antenna 138, a controller 136 for controlling one or more operations of the pump 150, a power supply 142, a safety system 146, and one or more indicators 135.

Since the display 154 is separable from the pump 150 and may not be coupled to the pump 150 when the pump 150 is carried and/or worn by the user, the pump 150 may include a limited user interface 137. The user interface 137 of the pump 150 may include one or more power buttons, one or more indicators, such as the indicator 135, and/or a pairing button (or other pairing feature) for pairing the pump 150 to the user device 130 and/or the display 154. The pump 150 may additionally and/or alternatively include an audio indicator for providing an alert and/or an alarm, a wireless system on chip (SOC) for motor and/or feedback control within the body of the pump 150, and a storage, which stores the fluid delivery protocols. Accordingly, the pump 150 may have a reduced profile, allowing the pump 150 to be carried and/or worn by the user, and limiting the amount of space occupied by the pump 150, such as when the pump 150 is carried and/or worn by the user. Such configurations additionally and/or alternatively allow the pump 150 to be located closer to the infusion site on the patient's body to provide more accurate measurements and reduce the likelihood that the pumps will be disconnected from the patient, improve the portability of the pump to allow patients to more comfortably use the pump away from the medical facility, and decrease the cost of manufacturing the pump 150.

To facilitate wearing, the pump 150 may include a clip, a clamp, strap, or other attachment fixture to couple the pump 150 to a garment or accessory (e.g., purse, belt, bag, hat, bicycle) worn by the patient. In some implementations, the pump 150 may include an adhesive element to semi-permanently attach the pump 150 to the patient or an item associated with the patient (e.g., purse, bag, clothing, etc.).

In some embodiments, the pump 150 includes a data storage 131, including one or more databases, data tables, and/or the like, for storing (e.g., temporarily storing) the fluid delivery protocol, one or more fluid parameters, one or more patient parameters, and/or one or more clinician parameters. In some implementations, the controller 136 of the pump 150 may verify the fluid delivery protocol, the one or more fluid parameters, the one or more patient parameters, and/or the one or more clinician parameters, such as a type of fluid to be delivered, a dosage of the fluid to be delivered, a delivery rate of the fluid to be delivered, a delivery rate of the fluid to be delivered, and/or the like. For example, the controller 136 may compare one or more stored fluid parameters to one or more of the patient parameters to determine whether the stored fluid parameters is the same as the stored patient parameters.

The pump 150 may include wireless communication circuitry 134 that is connected to and/or controlled by a controller 136. The wireless communication circuitry 134 may include one or more antennas 138 (which may include a near-field communication (NFC) antenna, a Bluetooth antenna, a Bluetooth Low Energy antenna, a Wi-Fi antenna, and/or other antennas), a barcode reader, and/or a radio frequency identification (RFID) tag reader. The antenna 138 may be configured to read from and/or write to a data tag positioned on or otherwise coupled to the display 154 and/or the user device 130. The data tag may be a type of wireless transceiver and may include a microcontroller unit (MCU), a memory, and an antenna (e.g., an NFC antenna) to perform the various functionalities described herein. The data tag may be, for example, a 1 Kbit or a 2 Kbit NFC tag. NFC tags with other specifications may also be used.

Additionally, and/or alternatively, the pump 150 may include communication circuitry 134 including one or more cellular communications features, such as via the network 105. The one or more cellular communications features may include General Packet Radio Services (GPRS), Long-Term Evolution (LTE) networks, 5G cellular technology, and/or the like. Such cellular communications features may increase system mobility and deployment options.

The wireless communication circuitry 134 may include additional components/circuitry for other communication modes, such as, for example, NFC circuitry, Bluetooth circuitry, Bluetooth Low Energy circuitry, and/or Wi-Fi circuitry and associated circuitry (e.g., control circuitry), for communication with other devices. For example, the pump 150 may be configured to wirelessly communicate with a remote processor (e.g., the display 154, the user device 130, a smartphone, a tablet, wearable electronics, a cloud server, and/or the like) through the wireless communication circuitry 134, and through this communication may receive and/or transmit one or more fluid parameters, one or more patient parameters, one or more clinician parameters, one or more fluid delivery protocols, and/or the like from and/or to one or more of the remote processors.

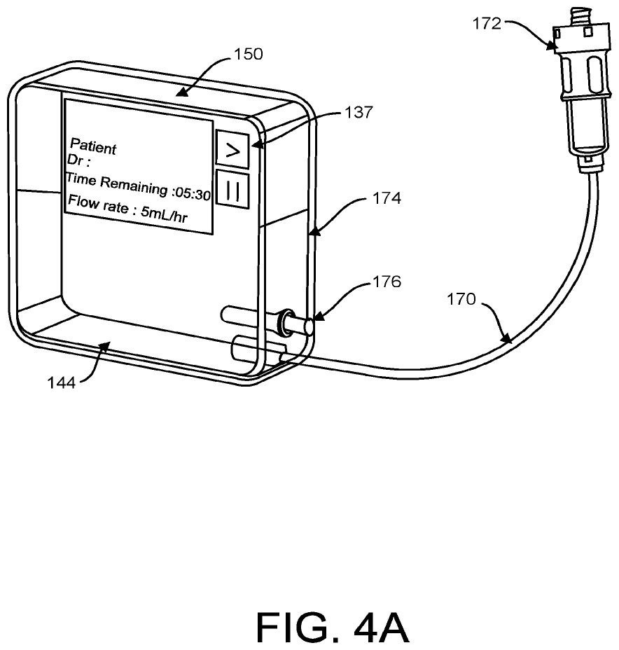

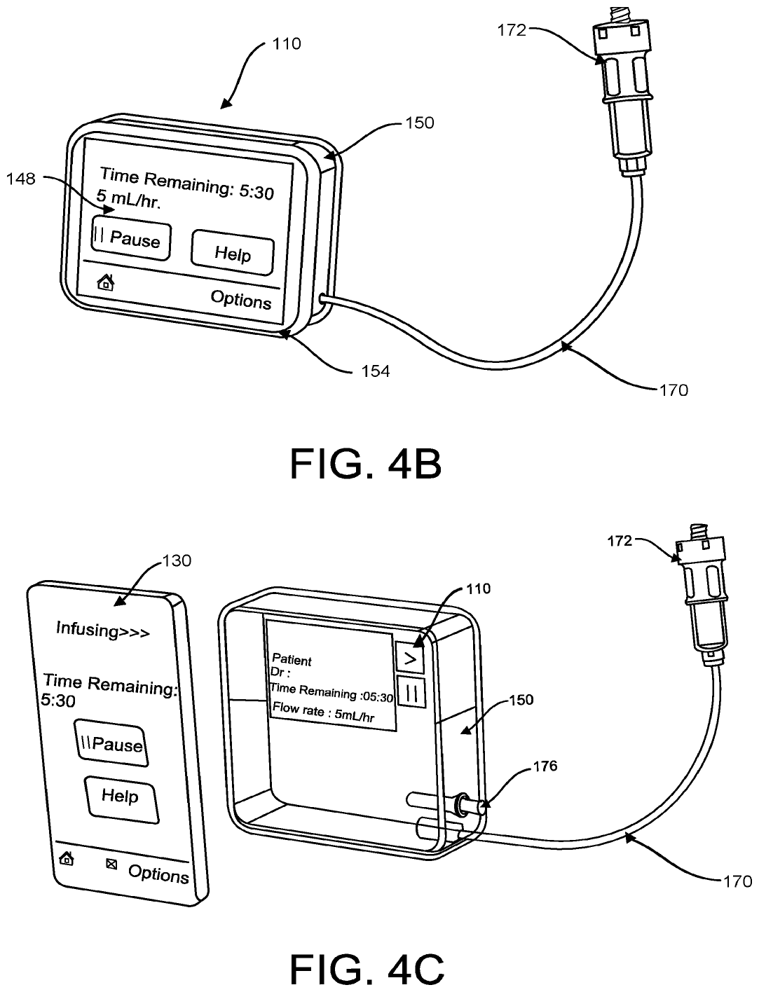

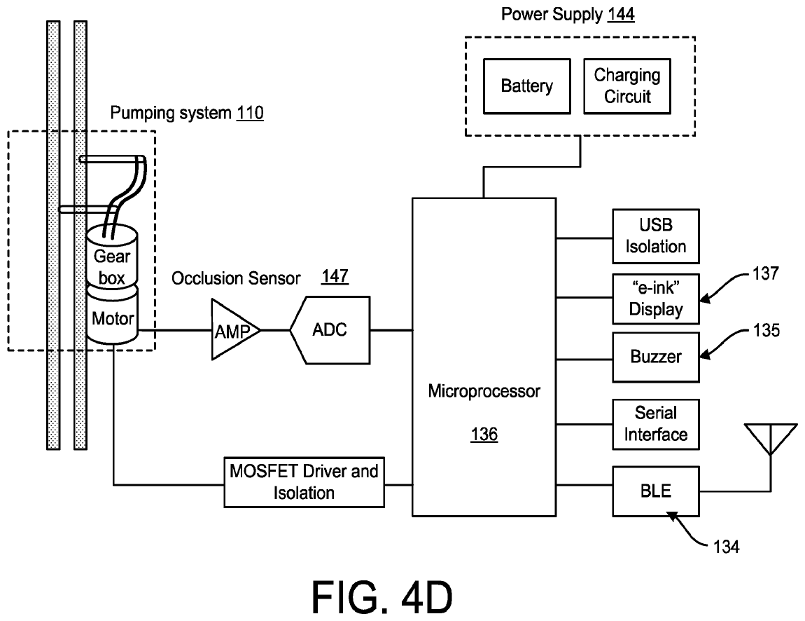

The fluid container 144 may store the fluid to be delivered by the pump 150 to the user. The fluid container 144 may be filled by a pharmacist, clinician, and/or the like, such as via a fill port 176 (see FIGS. 3A-3C and 4A-4C). As described herein, the fluid container 144 may be refillable (e.g., via the fill port 176), and/or may not be refillable such that the fluid container 144 along with the other components of the pump 150 are disposable after a single use or limited number of uses. In some implementations, the fluid container 144 may store a relatively large amount of fluid, since the pump 150 may be used for delivering fluid to the patient over an extended amount of time, and the pump 150 may be carried and/or worn by the patient throughout the duration of the delivery of therapy to the patient. For example, the fluid container may have a maximum capacity of approximately 150 mL, 250 mL, 100 to 150 mL, 150 to 200 mL, 200 to 250 mL, 250 to 300 mL, 300 to 350 mL, or greater. In some implementations, the fluid container may store one or more fluid bags that have a maximum capacity of approximately 150 mL, 250 mL, 100 to 150 mL, 150 to 200 mL, 200 to 250 mL, 250 to 300 mL, 300 to 350 mL, or greater.

The pumping mechanism 140 may drive the fluid from the fluid container 144 to be delivered to the patient via a fluid delivery tube. The pumping mechanism 140 may include a pump, motor, and/or the like. In some implementations, the pumping mechanism 140 includes a peristaltic pump mechanism (e.g., a linear type or a rotary type), a piston-type pump mechanism, and/or the like. The pumping mechanism 140 may allow viscous medications to be pumped efficiently, and improve the accuracy of the delivered fluid to the patient under varying temperature and pressure conditions. In some implementations, the pumping mechanism delivers the therapy to the patient over long periods of time. As such, in some instances, the pumping mechanism 140 may not need to be highly accurate and/or may be inexpensive.

In some implementations, after the pump 150 receives the desired fluid delivery protocol, the pump 150 may be used to deliver the fluid to the user based on the fluid delivery protocol. As described herein, the pump 150 may be used to deliver the fluid to the user at the medical facility and/or at locations remote from the medical facility, such as at the user's home and/or while the user is travelling.

The pump 150 may measure and/or log one or more parameters based on the fluid delivery protocol and/or the delivery of the fluid to the user. For example, the pump 150 may measure and/or log (e.g., store) one or more parameters, such as the time of the fluid delivery, the user's information, the number of fluid deliveries, the volume of fluid delivered, the type of fluid being delivered, the volume of the fluid remaining to be delivered, location data, and/or the like. The pump 150 may wirelessly transmit the measured and/or logged parameters to the user device 130 and/or to the display 154 (e.g., to the app). In some implementations, at least one of the measured and/or logged parameters may be displayed to the user via the display 154 and/or the user device 130. In some implementations, at least one of the measured and/or logged parameters may be transmitted from the user device 130 to a client device at the medical facility and/or directly to the server 126 to be stored in one or more of the data systems 125 described herein. Such configurations allow the pump 150 to be used at locations away from the medical facility, and still update the clinicians at the medical facility and/or the records associated with the delivery of the fluid therapy to the user.

In some implementations, the pump 150 may include a safety system 146 including one or more safety features. The safety features may include one or more sensors 147, an indicator 135, and an alert system 133. For example, the one or more sensors 147 may include one or more pressure sensors, flow sensors, and/or optical sensors that measure one or more aspects of the delivery of fluid to the patient, such as the volume of fluid delivered, the type of fluid being delivered, the volume of the fluid remaining to be delivered, the air level in the fluid delivery tube, and/or the like. The one or more sensors may additionally and/or alternatively include a battery sensor that detects the level of charge or remaining power capacity of the power supply 142. In some implementations, the pump 150, such as via the one or more safety features and/or as a result of one or more parameters measured by the one or more safety features, detect an error. The error may be associated with operation of the pump 150 and/or with the delivery of the fluid to the user.

For example, the pump 150 may detect a low battery, an occlusion in the fluid delivery line, a disconnection of the fluid delivery line from the user, a low volume of fluid remaining to be delivered, a malfunctioning component of the pump 150, a difference between the stored fluid parameter and the patient parameter, and/or the like. In response to detecting the error, the pump 150 may activate the alert system 133 and/or the indicator 135. The alert system 133 may include a visual, audio, and/or tactile indicator 135. For example, via the indicator 135, the alert system 133 may display visual (e.g., light, colored light such as a red, orange, yellow, or green light, a patterned light, and the like), audio (e.g., sound, patterned sound, and the like), and/or tactile (e.g., vibration, patterned vibration, and the like) feedback via the one or more of the indicators 135 on the pump 150. Additionally and/or alternatively, the pump 150 may transmit the error and/or details relating to the error to the display 154 and/or to the user device 130. The display 154 and/or the user device 130 may present the details associated with the error, to the user. The details relating to the error may include the type of error that has occurred, instructions for correcting the error, and/or instructions to contact the clinician. In some implementations, the display 154 and/or the user device 130 may contact the clinician in response to receiving the error and/or details associated with the error from the pump 150. For example, if the display 154 and/or the user device 130 is implemented as a smartphone or smart display, the details related to the error may cause the smartphone or smart display to initiate a communication session (e.g., chat, telephone call, voice over IP call, video call, etc.) with a device associated with the clinician.

In some implementations, in response to detection of the error, the pump 150 may stop delivery of the fluid to the user, for example, until the error is resolved (e.g., when the pump receives instructions to continue delivering the fluid). Additionally and/or alternatively, the display 154 and/or the user device 130 may cause the pump 150 to stop delivering the fluid to the user in response to receiving the error and/or details associated with the error from the pump 150. In some implementations, the display 154 and/or the user device 130 may cause the pump 150 to stop delivering the fluid to the user based on the type of error received from the pump 150. For example, if the display 154 and/or the user device 130 determines that the fluid delivery line is disconnected from the user and/or the fluid delivery line is occluded, the display 154 and/or the user device 130 may cause the pump 150 to sop delivering the fluid to the user. This allows for the pump 150 to be used at locations remote from the medical facility and improves the portability and usability of the pump 150 away from the medical facility.

Referring again to FIG. 2, the display 154 may form a part of the pump 150 and/or may be separately coupled to the pump 150. The display 154 may also include a user interface 148. The user interface 148 may form a part of a display screen of the display 154 that presents information to a user 160 (e.g., a patient or caregiver thereof such as a family member or home healthcare assistant) and/or the user interface may be separate from the display screen. For example, the user interface 148 may include one or more buttons, or portions of the display screen that is configured to receive an entry from the user 160. In some implementations, the display 154 (and/or the user interface of the pump 150) described herein displays information, such as a name of the fluid (e.g., the medication) to be delivered, and/or a volume of the fluid to be delivered, among other parameters of the fluid delivery protocol.

In some implementations, the display 154 includes communication circuitry 149. The communication circuitry 149 of the display 154 may communicate with the communication circuitry 134 of the pump 150, the user device 130, the server 126, and/or another device at the medical facility. The communication circuitry 149 may include an antenna, such as a near-field communication (NFC) antenna, a barcode reader, and/or a radio frequency identification (RFID) tag reader. The communication circuitry 149 may additionally and/or alternatively include a data tag. The data tag may be a type of wireless transceiver and may include a microcontroller unit (MCU), a memory, and an antenna (e.g., an NFC antenna) to perform the various functionalities described herein. The data tag may be, for example, a 1 Kbit or a 2 Kbit NFC tag. NFC tags with other specifications may also be used. The data tag may provide information, such as the fluid delivery protocol, to the pump 150.

Additionally, and/or alternatively, the communication circuitry 149 may include one or more cellular communications features, such as via the network 105. The one or more cellular communications features may include General Packet Radio Services (GPRS), Long-Term Evolution (LTE) networks, 5G cellular technology, and/or the like. Such cellular communications features may increase system mobility and deployment options. The communication circuitry 149 may include additional components/circuitry for other communication modes, such as, for example, Bluetooth, Bluetooth Low Energy, and/or Wi-Fi chips and associated circuitry (e.g., control circuitry), for communication with other devices, such as the pump 150, the user device 130, the server 126, and/or another device at the medical facility.

FIGS. 3A-3C illustrate an example of the infusion pump system 110, consistent with implementations of the current subject matter. As shown in FIGS. 3A-3C, and as described herein, the pump system 110 may be modular, including the portable infusion pump 150 and the display 154 that is separable from the pump 150. For example, FIG. 3A illustrates an example of the infusion pump system 110 in which the display 154 is coupled to the pump 150, such as via a magnetic connection, snap-fit arrangement, friction fit arrangement, mechanical fastener, and/or the like. When the display 154 is coupled to the pump 150 and/or held within a predetermined distance of the pump 150, the display 154 may transmit and/or receive data including the fluid delivery protocol, and/or one or more parameters associates with the patient, the delivery of the therapy, and the like.

As shown in FIG. 3A, the pump 150 may be coupled to a fluid delivery tube 170, which may couple to a drug delivery apparatus, such as a needle, via a connector 172 (e.g., a luer lock). The pump 150 may include one or more components, including the fluid container 144. For example, the fluid container 144 may hold the fluid to be delivered to the patient. In some implementations, the fluid container holds one or more (e.g., one, two, three, four, or more) fluid bags (e.g., IV bags), which may have a volume of up to or greater than 250 mL, less than or equal to 100 mL, 100 to 200 mL, 200 to 300 mL, 300 to 400 mL, 400 to 500 mL or greater. The fluid bags may be coupled to one end of the fluid delivery tube. 170. In such configurations, a clinician (e.g., the caregiver, pharmacist, and/or the like) may connect the fluid bags to the end of the fluid delivery tube. Alternatively, the fluid container 144 may be configured to hold the fluid to be delivered to the user. In such configurations, the fluid may flow directly between the interior of the fluid container 144 and the fluid delivery tube 170.

In some implementations, the pump 150 includes a power supply, such as the power supply 142. The power supply 142 may have a capacity of up to or greater than 7 days, including 1 to 2 days, 2 to 3 days, 3 to 4 days, 4 to 5 days, 5 to 6 days, 6 to 7 days, 1 to 2 weeks, or longer before recharging. Thus, the pump 150 may be used over an extended period of time and/or for multiple short periods of time as needed to deliver the fluid to the patient, depending on the fluid delivery protocol. To allow for the extended run time before recharging the power supply 142, the pump 150 may include low-power consuming components, such as a low-power consuming processor, a limited user interface, such as the user interface 137 and/or an e-link display, and communication circuitry 134 that includes Bluetooth communication.

In some implementations, the pump 150 includes a housing 174. The housing 174 may enclose the internal components of the pump 150. For example, the housing 174 encloses the internal components, such as the fluid container 144, the pumping mechanism 140, the communication circuitry 134, and/or the like. In some implementations, the housing 174 is waterproof, such that it allows for fluid protection up to or greater than IP66. This may allow for the user to use the pump 150 in the shower, and have no or minimal hindrance while traveling in rain, or in humid and/or dusty conditions.

In some implementations, the pump 150 includes an inductive coil and/or antenna, such as the antenna 138. The inductive coil may allow for NFC communication with the display 154, for example, although other communication methods, such as the methods described herein are contemplated. The NFC capabilities allows the pump 150 to pair with the display 154, and wirelessly transfer settings, such as one or more parameters and/or fluid delivery protocols, when the pump 150 and the display 154 contact one another and/or the display 154 is positioned within a distance from the pump 150. NFC also provides the capability to identify and communicate with a smart phone or tablet, such as the user device 130, to provide additional functionality allowing advanced programing and long distance monitoring by the clinician. For example, FIG. 3C illustrates an example of the infusion pump system 110 in which the user device 130 is communicatively coupled to the display 154 and/or the pump 150. The coil may additionally and/or alternatively allow for the power supply 142 of the pump 150 to be wirelessly charged (e.g., using Qi charging technology). Such configurations may reduce or eliminate externally-facing connection points on the pump 150 that may be otherwise susceptible for fluid ingress.

As described herein, the pump 150 may include a pumping mechanism 140. The pumping mechanism 140 may drive the fluid stored within the fluid container 144 and/or within the fluid bags from within the fluid container 144 to be delivered to the user. The pumping mechanism 140 may include a cassette-type interface with the pump 150, thus reducing the possibility of mis-loads. The cassette-type interface may include a peristaltic pump mechanism (e.g., a linear or rotary motor) to infuse the fluid to be delivered through the fluid delivery tube 170. Additionally and/or alternatively, the pumping mechanism 140 may include a piston-type mechanism that allows viscous fluids to be pumped efficiently, and improves the accuracy of the pumping mechanism 140 under varying temperature and pressure conditions.

In some implementations, the power supply 142 may be separated from the pump 150 for maintenance, recharging, and/or replacement. This allows for the pump to maintain a sufficient amount of power and manage battery life in an ambulatory setting. In some implementations, the one or more sensors 147 may detect when the power remaining in the power supply 142 is equal to or below a threshold amount (e.g., 5%, 10%, 15%, 20% capacity and/or the like). Upon determination of the limited power remaining, the controller 136 may cause the safety system 146 to issue an alert, such as via the indicator 135, the display 154, and/or the user deice 130. In some implementations, the alert includes an instruction to recharge or replace the power supply 142. Additionally and/or alternatively, upon detection of the low remaining power, the controller 136 may cause the pump to operate in a low-power or battery saving mode to ensure that the pump 150 has sufficient power to fully delivery the therapy to the patient. Additionally and/or alternatively, before beginning delivery of the fluid to the patient, the controller 136 may determine that the power supply 142 has an insufficient amount of power to begin delivery of the fluid to the patient and/or has an insufficient amount of power to last for the duration of the therapy based on the fluid delivery protocol transmitted from the display 154. In such instances, the controller 136 may cause the safety system 146 to issue an alert. In some implementations, the power supply 142 is coupled to the replaceable cassette-type pumping mechanism 140, which helps to ensure that the power supply 142 is fully charged with each refill of the fluid in the fluid container 144.

Thus, the pump 150 may allow for the minimally viable performance by delivering fluid to the user, without the display 154 coupled to the pump 150. Without the display 154 physically coupled to the pump 150, the pump 150 may be smaller in size, allowing it to be comfortably carried by the user (e.g., by way of a strap or belt clip), and affording more flexibility in performing normal daily activities. As an example, FIG. 3B illustrates the pump 150 separated from the display 154, consistent with implementations of the current subject matter.

FIGS. 4A-4D illustrate another example of the infusion pump system 110.

The example infusion pump system 110 shown in FIGS. 4A-4D may include the same or similar properties and/or features of the infusion pump system 110 shown in FIGS. 3A-3C. As described herein, the pump 150 may be separable from the display 154 (see FIG. 4B) and the display 154 and/or the user device 130 may wirelessly communicate with the pump 150 (see FIG. 4C). In the example infusion pump system 110 shown in FIGS. 4A-4D, the pump 150 may be disposable and/or form a disposable unit. The disposable pump 150 may help to reduce or eliminate mis-loading of the fluid delivery tubing onto the pumping mechanism 140 of the pump 150. For example, the fluid delivery tube 170 may be permanently coupled to the pump 150 or pumping mechanism 140 such that the fluid delivery tube 170 may not be separately coupled to the pump 150 during set up. This configuration helps to reduce issues resulting from mis-loading the fluid delivery tube, which may be more prominent in ambulatory settings.

Additionally and/or alternatively, the disposable pump 150 may reduce or eliminate maintenance, cleaning, calibration, testing, functional verification, and/or the like, which may otherwise be time consuming for the user of the pump 150. Such configurations may also reduce or eliminate the incidence of transfer of septic infections between multiple users when re-using a non-disposable pump.

Additionally and/or alternatively, the disposable pump 150 may include low cost materials. Although the low cost materials may not be durable, the disposable pumps 150 may be used for a limited amount of time (a single or limited number of doses). Thus, the pumps 150 may be discarded before the material of the pump 150 and the components thereof begins to wear. For example, the pump 150 may include one or more injection-molded plastic components, including a low-cost motor with plastic gearing, to reduce manufacturing costs. It should be appreciated that other materials, such as recycled plastic materials, may be used to form one or more components of the pump 150.

In some implementations, the pumping mechanism 140 is attached directly to a drive motor, eliminating the need for IV set interfaces. As shown in FIG. 4D, the electrical components of the pump 150 may use hard-wired connections, eliminating the cost of connectors and their associated reliability. Since the pump 150 may be disposable and thus, maintenance may not be performed on the pump 150, the entire electrical system of the pump 150 may be enclosed within a resin material to ensure protection of the internal components from fluid and impact damage from handling of the pump 150. Additionally and/or alternatively, the user interface 137 of the pump may use a low-energy material, such as e-paper.

Additionally and/or alternatively, the disposable pump 150 may enclose and/or entirely seal the internal components. This allows the pump 150 to be used in a non-sterile home environment or other ambulatory setting. As noted above, this also helps to prevent or limit ingress of unwanted fluids from entering the interior of the pump 150.

Additionally and/or alternatively, the disposable pump 150 provides users with updated software. For example, the pump 150 may include new and/or update software and/or firmware on the pump 150, as the pump 150 is disposed of and replaced. Such configurations save time for the consumer, and helps to ensure that the pump 150 has the most up-to-date software and/or firmware.

In some implementations, the disposable pump 150, such as the pump 150 shown in FIGS. 4A-4D, may be usable for a limited amount of time. For example, the pump 150 may be usable for a duration of approximately 30 days, 1 to 5 days, 5 to 10 days, 10 to 15 days, 15 to 20 days, 20 to 25 days, 25 to 30 days, 30 to 35 days, 35 to 40 days, or longer. In some implementations, the pump 150 may be usable for a length of time for which the included fluid is not expired. After the usable period lapses, the pump 150 may be disposed. In some implementations, the pump 150 may be disposed after a single use, after a single treatment, after a predetermined number of uses (e.g., one, two, three, four, or more uses), and/or after a predetermined number of treatments (e.g., one, two, three, four, or more treatments). Such configurations, as noted above, reduces maintenance or cleaning time.

In some implementations, the fluid container 144 may be integrated into the pump 150. For example, the fluid container 144 may directly store the fluid to be delivered to the user. This allows the fluid container 144 to be directly coupled to the fluid delivery tube 170. Such configurations also eliminate unneeded fitments and reduces the likelihood of infection, spillage, and/or leakage of fluids, such as hazardous medications. In some implementations, the pump 150 includes a fill port 176, which allows the fluid container 144 to be filled, such as at a pharmacy. In some implementations in which the pump 150 may be refilled a limited number of times, the fluid container 144 may be refilled via the fill port 176. The fill port 176 may include a sealed joint and check valve, or other valve that prevents or reduces spillage from the interior of the fluid container 144. In some implementations, the fluid container 144 may have a maximum capacity of approximately 150 mL, 250 mL, 100 to 150 mL, 150 to 200 mL, 200 to 250 mL, 250 to 300 mL, 300 to 350 mL, less, or greater. The disposable pump 150 may thus have a reduced cost and may improve the user experience while using the pump as the time for maintenance, servicing, and cleaning of the pump 150 would be reduced.

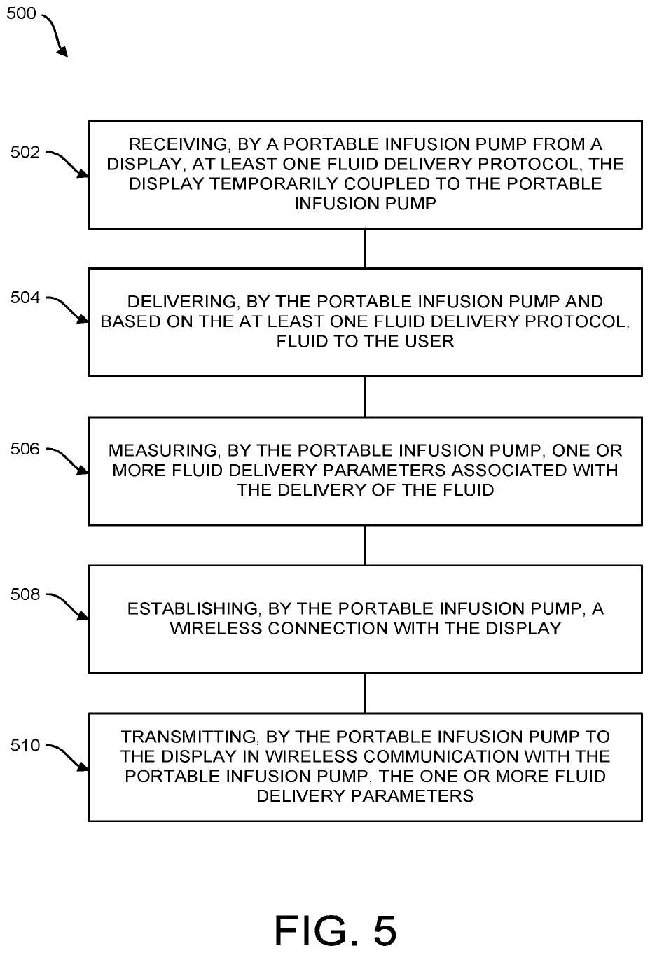

FIG. 5 illustrates an example process 500 for configuring a portable infusion pump, such as the pump 150, via a separable display, such as the display 154, consistent with implementations of the current subject matter. Consistent with implementations of the current subject matter, the infusion pump system including the pump 150 and/or the display 154, may reduce the amount of space pumps occupy in a medical facility, allow the pump to be located closer to the infusion site on the patient's body to provide more accurate measurements and reduce the likelihood that the pump will be disconnected from the patient, improve the portability of the pump to allow patients to more comfortably use the pumps away from the medical facility, decrease the cost of manufacturing the pump, and/or the like.

In some implementations, as described herein, the display may be physically coupled (e.g., in contact with) the portable infusion pump and/or the display may be held within a predetermined distance from the portable infusion pump to establish a wireless connection with the pump. At 502, the portable infusion pump may receive, from the display, at least one fluid delivery protocol. For example, the portable infusion pump may receive, via NFC, Bluetooth, and/or the like, the at least one fluid delivery protocol. The at least one fluid delivery protocol may include one or more parameters for delivering a fluid to a user. For example, the one or more parameters may include a volume of fluid to be delivered to the user, a type of fluid to be delivered to the user, a time interval of fluid delivery, a frequency of fluid delivery, a delivery rate at which the fluid is to be delivered to the user, and/or the like.

As described above with respect to at least FIGS. 1-4D, the display may be temporarily coupled to the portable infusion pump, and may be separated from the portable infusion pump after transmission of the fluid delivery protocol from the display to the portable infusion pump. After the display is removed from the portable infusion pump, the portable infusion pump may include limited functionality. For example, the portable infusion pump may include an indicator for providing visual, tactile, and/or audio feedback to the user based on one or more operations of the portable infusion pump. The indicator may display a light, change a light color, change a light pattern, flash a light, play a sound, play a sound pattern, play a vibrational pattern, and/or the like, when an error occurs in the delivery of the fluid to the user. The error may indicate an occlusion in the fluid delivery line, air is present within the fluid delivery tube, an interruption in the connection between the fluid delivery line and the user, a low battery of the portable infusion pump, a low volume of fluid to be delivered to the user and/or the like. Such configurations help the portable infusion pump maintain a reduced profile to improve portability and may allow for the portable infusion pump to be carried and/or worn by the user.

At 504, the fluid may be delivered, based on the at least one fluid delivery protocol, by the portable infusion pump to the user. For example, the portable infusion pump may deliver the fluid to the user based on the fluid delivery rate, the time intervals, and/or the like stored as part of the fluid delivery protocol.

At 506, the portable infusion pump may measure and/or log one or more fluid delivery parameters associated with the delivery of the fluid by the portable infusion pump. For example, the portable infusion pump may measure and/or log the time of the fluid delivery, the user's information, the number of fluid deliveries, the volume of fluid delivered, the type of fluid being delivered, the volume of the fluid remaining to be delivered, location data, and/or the like.

At 508, the portable infusion pump may establish and/or re-establish a wireless connection with the display. For example, the pump may be brought into contact with and/or within a predetermined distance from the display, as described herein.

At 510, the portable infusion pump may transmit the measured and/or logged fluid delivery parameters associated with the delivery of the fluid to the display for display to the user. In some implementations, the display (e.g., via an app) displays at least one of the measured and/or logged fluid delivery parameters to the user and/or transmits the measured and/or logged fluid delivery parameters to the clinician. In some implementations, the portable infusion pump may detect an error. In response to detecting the error, the portable infusion pump may provide visual, tactile, and/or audio feedback to the user via the indicator on the portable infusion pump and/or the display, and/or transmit the error to the display. The display may then transmit the error and/or information associated with the error to the clinician. In some implementations, the portable infusion pump stops the delivery of the fluid to the user in response to detecting the error.

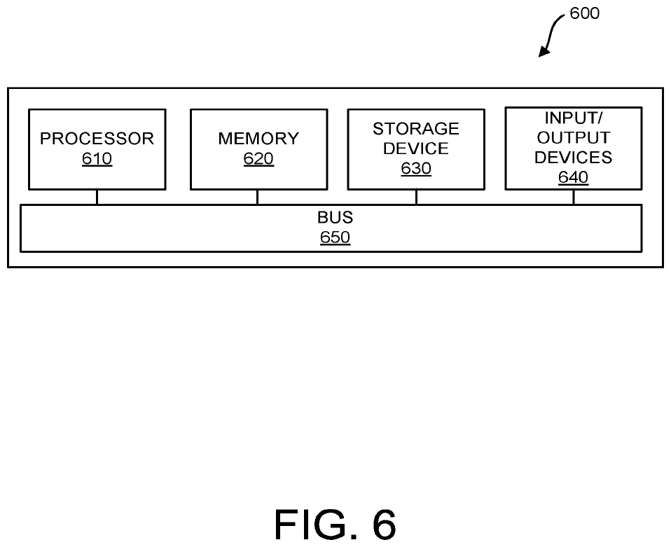

FIG. 6 depicts a block diagram illustrating a computing system 600 consistent with implementations of the current subject matter. Referring to FIGS. 1 and 6, the computing system 600 can be used to implement the infusion pump system 110 including the portable infusion pump 150 and the display 154, the data systems 125, the server 126, the user device 130, and/or any components therein.

As shown in FIG. 6, the computing system 600 can include a processor 610, a memory 620, a storage device 630, and input/output devices 640. The processor 610, the memory 620, the storage device 630, and the input/output devices 640 can be interconnected via a system bus 650. The processor 610 is capable of processing instructions for execution within the computing system 600. Such executed instructions can implement one or more components of, for example, the display 154 and/or the portable infusion pump 150. In some example embodiments, the processor 610 can be a single-threaded processor. Alternatively, the processor 610 can be a multi-threaded processor. The processor 610 is capable of processing instructions stored in the memory 620 and/or on the storage device 630 to present graphical information for a user interface provided via the input/output device 640.

The memory 620 is a computer readable medium such as volatile or non-volatile that stores information within the computing system 600. The memory 620 can store data structures representing configuration object databases, for example. The storage device 630 is capable of providing persistent storage for the computing system 600. The storage device 630 can be a floppy disk device, a hard disk device, an optical disk device, or a tape device, or other suitable persistent storage means. The input/output device 640 provides input/output operations for the computing system 600. In some example embodiments, the input/output device 640 includes a keyboard and/or pointing device. In various implementations, the input/output device 640 includes a display unit for displaying graphical user interfaces.

According to some example embodiments, the input/output device 640 can provide input/output operations for a network device. For example, the input/output device 640 can include Ethernet ports or other networking ports to communicate with one or more wired and/or wireless networks (e.g., a local area network (LAN), a wide area network (WAN), the Internet).

In some example embodiments, the computing system 600 can be used to execute various interactive computer software applications that can be used for organization, analysis and/or storage of data in various formats. Alternatively, the computing system 600 can be used to execute software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 640. The user interface can be generated and presented to a user by the computing system 600 (e.g., on a computer screen monitor, etc.).

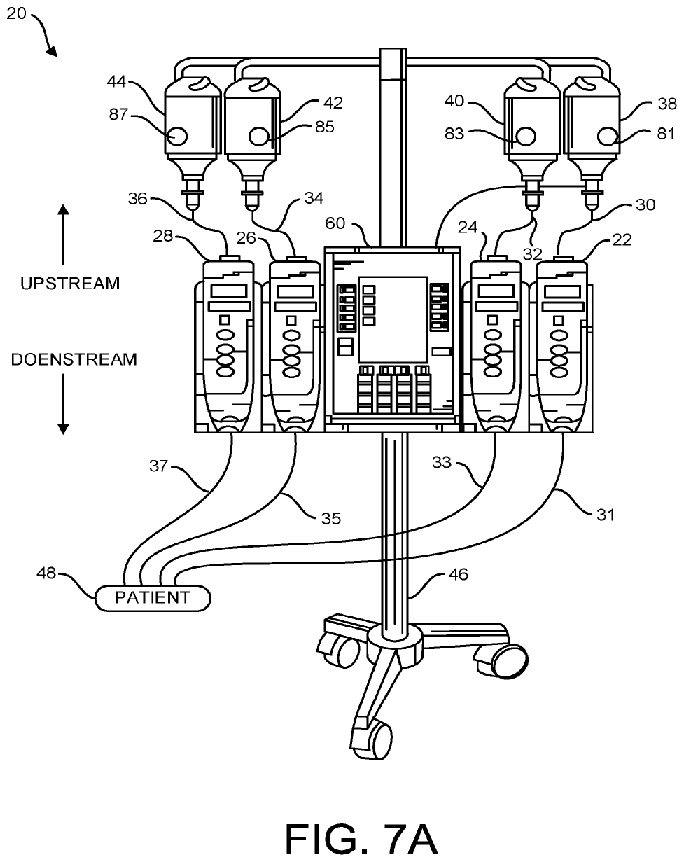

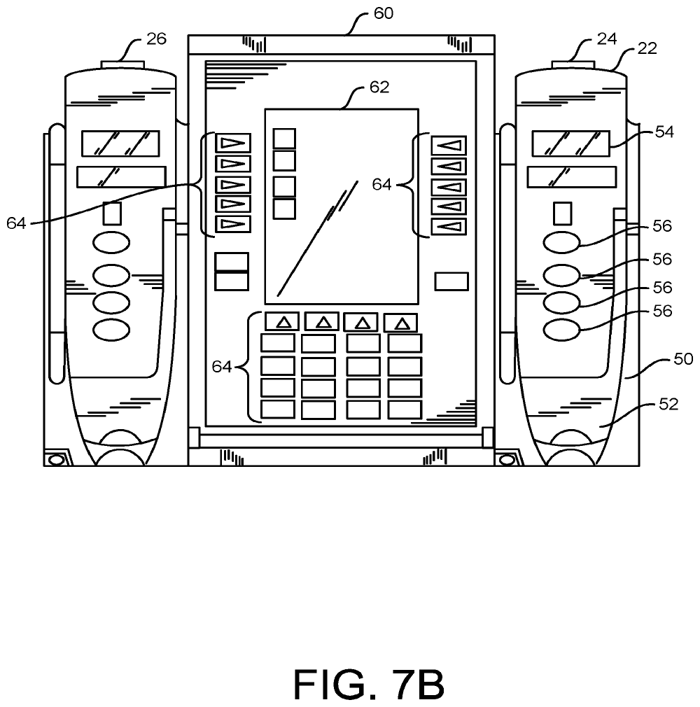

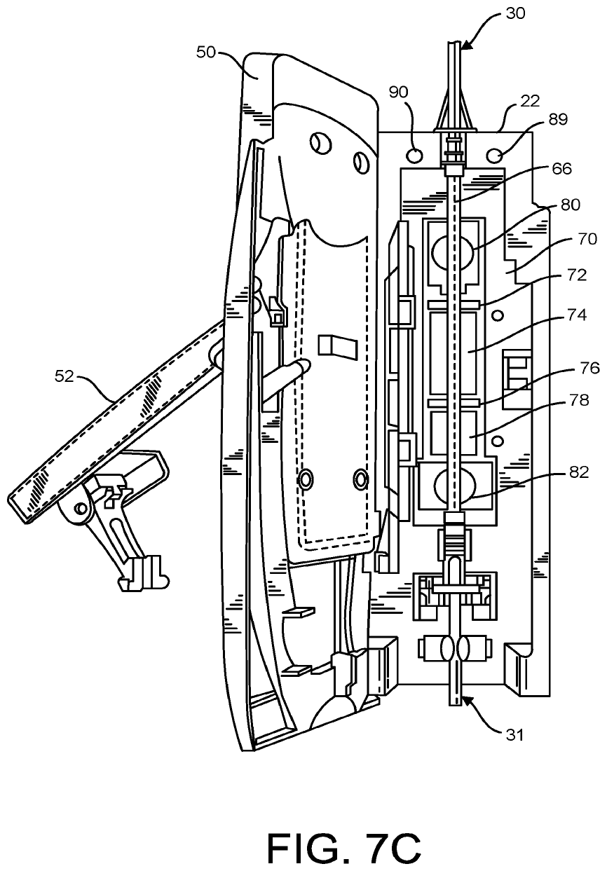

In some example embodiments, a pump 22 (e.g., the portable infusion pump 150) may be part of a patient care system 20. FIGS. 7A-7C illustrate example embodiments of the patient care system 20, though other types of patient care systems may be implemented. Referring to FIG. 7A, the patient care system 20 may include the pump 22 as well as additional pumps 24, 26, and 28. Although a large volume pump (LVP) is illustrated, other types of pumps may be implemented, such as a small volume pump (SVP), a TCI pump, a syringe pump, an anesthesia delivery pump, and/or a patient-controlled analgesic (PCA) pump configured to deliver a medication to a patient in accordance with one or more fluid delivery protocols. In some implementations, the portable infusion pump 150 described herein may be communicatively coupled with and/or form a part of the pumps 22, 24, 26, 28. The pump 22 may be any infusion device configured to deliver a substance (e.g., fluid, nutrients, medication, and/or the like) to a patient's circulatory system or epidural space via, for example, intravenous infusion, subcutaneous infusion, arterial infusion, epidural infusion, and/or the like, or the pump 22 may be an infusion device configured to deliver a substance (e.g., fluid, nutrients, medication, and/or the like) to a patient's digestive system via a nasogastric tube (NG), a percutaneous endoscopic gastrostomy tube (PEG), nasojejunal tube (NJ), and/or the like.

As shown in FIG. 7A, each of the pump 22, 24, 26, and 28 may be fluidly connected with an upstream fluid line 30, 32, 34, and 36, respectively. Moreover, each of the four pumps 22, 24, 26, and 28 may also fluidly connected with a downstream fluid line 31, 33, 35, and 37, respectively. The fluid lines can be any type of fluid conduit, such as tubing, through which fluid can flow. At least a portion of one or more of the fluid lines may be constructed with a multi-layered configuration as described herein.

Fluid supplies 38, 40, 42, and 44, which may take various forms but in this case are shown as bottles, are inverted and suspended above the pumps. Fluid supplies may also take the form of bags, syringes, or other types of containers. Both the patient care system 20 and the fluid supplies 38, 40, 42, and 44 may be mounted to a roller stand or intravenous (IV) pole 46.

A separate pump 22, 24, 26, and 28 may be used to infuse each of the fluids of the fluid supplies into the patient. The pumps 22, 24, 26, and 28 may be flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may comprise drugs or nutrients or other fluids.

Typically, medical fluid administration sets have more parts than are shown in FIG. 7A. Many have check valves, drip chambers, valved ports, connectors, and other devices well known to those skilled in the art. These other devices have not been included in the drawings so as to preserve clarity of illustration. In addition, it should be noted that the drawing of FIG. 7A is not to scale and that distances have been compressed for the purpose of clarity. In an actual setting, the distance between the bottles 38, 40, 42, and 44 and the pump modules 22, 24, 26, and 28 could be much greater.

Referring now to FIG. 7B, an enlarged view of the front of the patient care system 20 is shown. The pump 22 may include a front door 50 and a handle 52 that operates to lock the door in a closed position for operation and to unlock and open the door for access to the internal pumping and sensing mechanisms and to load administration sets for the pump. When the door is open, the tube can be connected with the pump, as will be shown in FIG. 7C. When the door is closed, the tube is brought into operating engagement with the pumping mechanism, the upstream and downstream pressure sensors, and the other equipment of the pump. A display 54, such as an LED display, is located in plain view on the door in this embodiment and may be used to visually communicate various information relevant to the pump, such as alert indications (e.g., alarm messages), such as via the safety system 146. The display 54 may otherwise be a part of or be coupled to the pump 22. Control keys 56 exist for programming and controlling operations of the pump as desired. The pump 22 also includes audio alarm equipment in the form of a speaker (not shown).

In the embodiment shown, a programming module 60 is attached to the left side of the pump 22. In some embodiments, the programming module 60 forms a part of the pump 22. Other devices or modules, including another pump, may be attached to the right side of the pump 22, as shown in FIG. 7A. In such a system, each attached pump represents a pump channel of the overall patient care system 20. In one embodiment, the programming module is used to provide an interface between the pump 22 and external devices as well as to provide most of the operator interface for the pump 22.

The programming module 60 includes a display 62 for visually communicating various information, such as the operating parameters of the pump 22 and alert indications and alarm messages. In some implementations, the display 62 forms a part of or is in communication with the portable infusion pump 150, the display 154, and/or the user device 130. The programming module 60 may additionally and/or alternatively display the one or more patient parameters and the corresponding values for each of the one or more patient parameters described herein to the display 54 and/or the display 64. The programming module 60 may also include a speaker to provide audible alarms. The programming module or any other module also has various input devices in this embodiment, including control keys 64 and a bar code or other scanner or reader for scanning information from an electronic data tag relating to the infusion, the patient, the care giver, or other. The programming module also has a communications system (not shown) with which it may communicate with external equipment such as a medical facility server or other computer and with a portable processor, such as a handheld portable digital assistant (“PDA), or a laptop-type of computer, or other information device that a care giver may have to transfer information as well as to download drug libraries to a programming module or pump. In some embodiments, the pump 22 may communicate with a server (e.g., the server 126) and/or one or more portable infusion pumps 150 to transmit one or more fluid delivery protocols to the one or more portable infusion pumps 150.

In some implementations, the programming module 60 may be configured as the display for the portable infusion pump 150. The programming module 60 may pair with the portable infusion pump 150 using a proprietary or standards based device pairing protocol such as Bluetooth or WiFi connection protocols. Once paired, information received by the programming module 60 may be transmitted to the portable infusion pump 150. Conversely, alerts or alarms generated by the portable infusion pump 150 may be transmitted to the programming module 60 which may, in turn, present a human perceivable manifestation of the alert or alarm using one or more output devices included in the programming module 60. Examples of the output devices for presenting the manifestation include audio output device, illumination device (e.g., LED), or a display (e.g., graphical user interface).

The communications system may take the form of a radio frequency (“RF”) (radio frequency) system, an optical system such as infrared, a Bluetooth system, or other wired or wireless system. The bar code scanner and communications system may alternatively be included integrally with the pump 22, such as in cases where a programming module is not used, or in addition to one with the programming module. Further, information input devices need not be hard-wired to medical instruments, information may be transferred through a wireless connection as well.

FIG. 7B includes a second pump 26 connected to the programming module 60. As shown in FIG. 7A, more pump modules may be connected. Additionally, other types of modules may be connected to the pump modules or to the programming module. In such implementations, the programming module may maintain determine, adjust, and/or display values (e.g., default values) of each of the one or more patient parameters for each pump (e.g., pump 22 and pump 26).

Turning now to FIG. 7C, the pump 22 is shown in perspective view with the front door 50 open, showing the upstream fluid line 30 and downstream fluid line 31 in operative engagement with the pump 22. The pump 22 directly acts on a tube 66 (also referred to as a pump segment) that connects the upstream fluid line 30 to the downstream fluid line 31 to form a continuous fluid conduit, extending from the respective fluid supply 38 (FIG. 7A) to the patient 48, through which fluid is acted upon by the pump to move fluid downstream to the patient. Specifically, a pumping mechanism 70 acts as the flow control device of the pump to move fluid though the conduit. The upstream and downstream fluid lines and/or tube 66 may be coupled to a pump cassette or cartridge that is configured to be coupled to the pump 22, such as the type described in co-pending U.S. patent application Ser. No. 13/827,775, which is incorporated by reference herein.

The type of pumping mechanism, such as the pumping mechanism 140, may vary and may be for example, a multiple finger pumping mechanism. For example, the pumping mechanism may be of the “four finger” type and includes an upstream occluding finger 72, a primary pumping finger 74, a downstream occluding finger 76, and a secondary pumping finger 78. The “four finger” pumping mechanism and mechanisms used in other linear peristaltic pumps operate by sequentially pressing on a segment of the fluid conduit by means of the cam-following pumping fingers and valve fingers 72, 74, 76, and 78. The pressure is applied in sequential locations of the conduit, beginning at the upstream end of the pumping mechanism and working toward the downstream end. At least one finger is always pressing hard enough to occlude the conduit. As a practical matter, one finger does not retract from occluding the tubing until the next one in sequence has already occluded the tubing; thus at no time is there a direct fluid path from the fluid supply to the patient. The operation of peristaltic pumps including four finger pumps is well known to those skilled in the art and no further operational details are provided here.

In this particular embodiment, FIG. 7C further shows a downstream pressure sensor 82 included in the pump 22 at a downstream location with respect to the pumping mechanism. The downstream pressure sensor 82 is mounted to the flow control device 70 and is located adjacent and downstream in relation to the flow control device. The downstream pressure sensor is located downstream from the flow control device, that is, at a location between the patient 48 (FIG. 7A) and the flow control device, so that the connection of the correct fluid supply with the correct pump may be verified before any fluid is pumped to the patient.

With reference still to FIG. 7C, an upstream pressure sensor 80 may also be included in the pump 22. The upstream pressure sensor is assigned to the flow control device or pumping mechanism 70 and, in this embodiment, is further provided as an integral part of the pump 22. It is mounted to the flow control device 70 and is located adjacent and upstream in relation to the flow control device. The upstream pressure sensor is located upstream from the flow control device, that is, at a location between the fluid supply 38 (FIG. 7A) and the flow control device, so that the connection of the correct fluid supply with the correct pump may be verified before any fluid is pumped to the patient. In an implementation where the source is a syringe, the flow control device 70 may be configured to press a plunger of the syringe to provide the infusion according to the programmed parameters.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

Although the disclosure, including the figures, described herein may describe and/or exemplify different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An infusion pump system, comprising:
a portable infusion pump configured to be worn by a user, the portable infusion pump comprising:
a fluid container configured to store a fluid to be delivered to the user;
a pumping mechanism configured to drive the delivery of the fluid from the fluid container to the user via a fluid delivery tube; and
communications circuitry, the communications circuitry comprising an antenna; and
a separable display configured to transmit, to the communications circuitry of the portable infusion pump when the separable display is coupled to the portable infusion pump, a fluid delivery protocol, the separable display further configured to be separated from the portable infusion pump after transmission of the fluid delivery protocol to the portable infusion pump, the fluid delivery protocol defining one or more parameters for delivering the fluid to the user.

2. The system of claim 1, wherein the portable infusion pump is disposable.

3. The system of any one of claims 1 to 2, wherein the communications circuitry comprises at least one of a low energy communications circuitry, a near field communications circuitry, and a Bluetooth low energy communications circuitry.

4. The system of any one of claims 1 to 3, wherein the separable display includes an attachment sensor, and wherein the separable display is configured to transmit the fluid delivery protocol when a signal from the attachment sensor indicates that the separable display is physically coupled to the portable infusion pump.

5. The system of claim 4, wherein the physical coupling comprises a magnetic coupling.

6. The system of any one of claims 1 to 3, wherein the separable display includes a proximity sensor, and wherein the separable display is configured to transmit the fluid delivery protocol when a signal from the proximity sensor indicates that the separable display is positioned within a predetermined distance from the portable infusion pump.

7. The system of any one of claims 1 to 6, further comprising the fluid delivery tube.

8. The system of any one of claims 1 to 7, wherein the portable infusion pump includes a waterproof housing, and wherein at least the fluid container, the pumping mechanism, and the communication circuitry are disposed within the waterproof housing.

9. The system of any one of claims 1 to 8, wherein the portable infusion pump comprises an alert system for generating an alert upon detection of an error during the delivery of the fluid to the user, the alert comprising one or more of an audio, visual, and tactile alert.

10. The system of any one of claims 1 to 9, wherein the fluid container is configured to hold less than or equal to 100 milliliters of the fluid.

11. A portable infusion pump configured to be worn by a user, the portable infusion pump comprising:
a fluid container configured to store a fluid to be delivered to the user;
a pumping mechanism configured to drive the delivery of the fluid from the fluid container to the user via a fluid delivery tube; and
communications circuitry, the communications circuitry comprising an antenna;
wherein the portable infusion pump is configured to be temporarily coupled to a separable display;
wherein the communications circuitry is configured to receive, from the separable display, a fluid delivery protocol, when the portable infusion pump is coupled to the separable display, the fluid delivery protocol defining one or more parameters for delivering the fluid to the user; and
wherein the portable infusion pump is configured to be separated from the display after receiving, from the display, the fluid delivery protocol.

12. The pump of claim 11, wherein the portable infusion pump is disposable.

13. The pump of any one of claims 11 to 12, wherein the communications circuitry comprises at least one of a low energy communications circuitry, a near field communications circuitry, and a Bluetooth low energy communications circuitry.

14. The pump of any one of claims 11 to 13, wherein the separable display includes an attachment sensor, and wherein the separable display is configured to transmit the fluid delivery protocol when a signal from the attachment sensor indicates that the separable display is physically coupled to the portable infusion pump.

15. The pump of claim 14, wherein the physical coupling comprises a magnetic coupling.

16. The pump of any one of claims 11 to 13, wherein the separable display includes a proximity sensor, and wherein the separable display is configured to transmit the fluid delivery protocol when a signal from the proximity sensor indicates that the separable display is positioned within a predetermined distance from the portable infusion pump.

17. The pump of any one of claims 11 to 16, further comprising a fill port, through which the fluid container is configured to be filed.

18. The pump of any one of claims 11 to 17, wherein the fluid container is configured to hold a plurality of fluid bags.

19. The pump of any one of claims 11 to 18, wherein the portable infusion pump includes is a waterproof housing, and wherein at least the fluid container, the pumping mechanism, and the communication circuitry are disposed within the waterproof housing.

20. The pump of any one of claims 11 to 19, further comprising an alert system for generating an alert upon detection of an error during the delivery of the fluid to the user, the alert comprising one or more of an audio, visual, and tactile alert.

21. The pump of any one of claims 11 to 20, wherein the fluid container is configured to hold less than or equal to 100 milliliters of the fluid.

22. An infusion management system, comprising:
at least one data processor; and
at least one memory storing instructions which, when executed by the at least one data processor, result in operations comprising:
receiving, by a portable infusion pump from a display, at least one fluid delivery protocol, the display being temporarily coupled to the portable infusion pump;
delivering, by the portable infusion pump and based on the at least one fluid delivery protocol, a fluid to a user;
measuring, by the portable infusion pump, one or more fluid delivery parameters associated with the delivery of the fluid;
establishing, by the portable infusion pump, a wireless connection with the display; and
transmitting, by the portable infusion pump to the display in wireless communication with the portable infusion pump, the one or more fluid delivery parameters.

23. The system of claim 22, wherein the operations further comprise:
detecting an error in the delivery of the fluid to the user; and
stopping the delivery of the fluid to the user.

24. The system of claim 23, wherein the operations further comprise:
transmitting the error to the display when the display is in wireless communication with the portable infusion pump; and
displaying information associated with the error via the display.

25. The system of any one of claims 22 to 24, wherein the portable infusion pump comprises:
a fluid container configured to store the fluid to be delivered to the user;
a pumping mechanism configured to drive the delivery of the fluid from the fluid container to the user via a fluid delivery tube; and
communications circuitry, the communications circuitry comprising an antenna.

26. The system of any one of claims 22 to 25, wherein the operations further comprise:
establishing, by the portable infusion pump, a wireless connection with the display when an attachment sensor of the display indicates that portable infusion pump is physically coupled to the display; and
disconnecting, by the portable infusion pump, the wireless connection with the display when the portable infusion pump is not physically coupled to the display.

27. The system of any one of claims 22 to 26, wherein the portable infusion pump is disposable.

28. The system of claim 25, wherein the communications circuitry comprises at least one of a low energy communications circuitry, a near field communications circuitry, and a Bluetooth low energy communications circuitry.

29. The system of claim 25, wherein the portable infusion pump further comprises a fill port, through which the fluid container is configured to be filed.

30. The system of claim 25, wherein the fluid container is configured to hold a plurality of fluid bags.

31. The system of any one of claims 22 to 30, wherein the portable infusion pump includes a waterproof housing, and wherein at least the fluid container, the pumping mechanism, and the communication circuitry are disposed within the waterproof housing.

32. The system of any one of claims 25 to 31, wherein the portable infusion pump further comprises an alert system for generating an alert upon detection of an error during the delivery of the fluid to the user, the alert comprising one or more of an audio, visual, and tactile alert.

33. The system of any one of claims 25 to 32, wherein the fluid container is configured to hold less than or equal to 100 milliliters of the fluid.

34. A method, comprising:
receiving, by a portable infusion pump from a display, at least one fluid delivery protocol, the display being temporarily coupled to the portable infusion pump;
delivering, by the portable infusion pump and based on the at least one fluid delivery protocol, a fluid to a user;
measuring, by the portable infusion pump, one or more fluid delivery parameters associated with the delivery of the fluid;
establishing, by the portable infusion pump, a wireless connection with the display; and
transmitting, by the portable infusion pump to the display in wireless communication with the portable infusion pump, the one or more fluid delivery parameters.

35. The method of claim 34, further comprising:
detecting an error in the delivery of the fluid to the user; and
stopping the delivery of the fluid to the user.

36. The method of claim 35, further comprising:
transmitting the error to the display when the display is in wireless communication with the portable infusion pump; and
displaying information associated with the error via the display.

37. The method of any one of claims 34 to 36, wherein the portable infusion pump comprises:
a fluid container configured to store the fluid to be delivered to the user;
a pumping mechanism configured to drive the delivery of the fluid from the fluid container to the user via a fluid delivery tube; and
communications circuitry, the communications circuitry comprising an antenna.

38. The method of any one of claims 34 to 37, further comprising:
establishing, by the portable infusion pump, a wireless connection with the display when an attachment sensor of the display indicates that portable infusion pump is physically coupled to the display; and
disconnecting, by the portable infusion pump, the wireless connection with the display when the portable infusion pump is not physically coupled to the display.

39. The method of any one of claims 34 to 38, wherein the portable infusion pump is disposable.

40. The method of claim 37, wherein the communications circuitry comprises at least one of a low energy communications circuitry, a near field communications circuitry, and a Bluetooth low energy communications circuitry.

41. The method of claim 37, wherein the portable infusion pump further comprises a fill port, through which the fluid container is configured to be filed.

42. The method of claim 37, wherein the fluid container is configured to hold a plurality of fluid bags.

43. The method of any one of claims 34 to 42, wherein the portable infusion pump includes a waterproof housing, and wherein at least the fluid container, the pumping mechanism, and the communication circuitry are disposed within the waterproof housing.

44. The method of any one of claims 34 to 43, wherein the portable infusion pump further comprises an alert system for generating an alert upon detection of an error during the delivery of the fluid to the user, the alert comprising one or more of an audio, visual, and tactile alert.

45. The method of any one of claims 34 to 44, wherein the fluid container is configured to hold less than or equal to 100 milliliters of the fluid.

46. A non-transitory computer-readable storage medium including program code, which when executed by at least one data processor, cause operations comprising:
receiving, by a portable infusion pump from a display, at least one fluid delivery protocol, the display being temporarily coupled to the portable infusion pump;
delivering, by the portable infusion pump and based on the at least one fluid delivery protocol, a fluid to a user;
measuring, by the portable infusion pump, one or more fluid delivery parameters associated with the delivery of the fluid;
establishing, by the portable infusion pump, a wireless connection with the display; and
transmitting, by the portable infusion pump to the display in wireless communication with the portable infusion pump, the one or more fluid delivery parameters.

47. An apparatus, comprising:
means for receiving, by a portable infusion pump from a display, at least one fluid delivery protocol, the display being temporarily coupled to the portable infusion pump;
means for delivering, by the portable infusion pump and based on the at least one fluid delivery protocol, a fluid to a user;
means for measuring, by the portable infusion pump, one or more fluid delivery parameters associated with the delivery of the fluid;
means for establishing, by the portable infusion pump, a wireless connection with the display; and
means for transmitting, by the portable infusion pump to the display in wireless communication with the portable infusion pump, the one or more fluid delivery parameters.

48. The apparatus of claim 47, comprising:
means for performing any of the functions recited in any of claims 35 to 45.
